(19) United States
(12) Patent Application Publication
(10) Pub. No.: US 2022/0274113 A1
(43) Pub. Date: Sep. 1, 2022

(54) APPARATUS AND METHODS FOR MANIPULATING MICRODROPLETS

(71) Applicant: Lightcast Discovery Ltd., Cambridge (GB)

(72) Inventors: Cameron FRAYLING, Cambridge (GB); Thomas Henry ISAAC, Cambridge (GB); Maciej SOSNA, Cambridge (GB); Evangelia-Nefeli ATHANASOPOULOU, Cambridge (GB)

(73) Assignee: Lightcast Discovery Ltd., Cambridge (GB)

(21) Appl. No.: 17/626,230

(22) PCT Filed: Jul. 10, 2020
(86) PCT No.: PCT/GB2020/051665

§ 371 Date: Jan. 11, 2022

(30) Foreign Application Priority Data
Jul. 12, 2019 (GB) 1910035.3

(51) Int. Cl. B01L 3/00; C12M 3/06; C12M 1/00

(52) U.S. Cl. CPC B01L 3/502792; C12M 23/16; C12M 23/20; B01L 2200/0652; B01L 2300/163; B01L 2300/168

(57) ABSTRACT

A device for manipulating microdroplets, the device comprising a microfluidic chip adapted to receive and manipulate microdroplets dispersed in carrier fluid flowing along pathways on a surface of the chip, wherein the microdroplets are manipulated using an optically-mediated electrowetting (oEWOD) force; characterised in that the surface of the chip comprises a coating structure configured to allow controlled attachment and/or detachment of adherent cells contained within the microdroplets by application of the oEWOD force.

### FIELD

The present disclosure relates to a device and associated methods for manipulating microdroplets, and in particular to a microfluidic chip comprising a coating structure, the microfluidic chip configured to manipulate microdroplets and to allow controlled attachment and detachment of adherent cells contained within the microdroplets by application of oEWOD force.

### BACKGROUND

Cells derived from animal tissues can be manipulated in culture for use as a research tool, for the production of virus vaccines and various therapeutic proteins, and to generate functional cells or tissue analogues for screening of medicines.

Mammalian cells can be made to produce vaccines through viral infection, and therapeutic proteins through genetic engineering. Many of these medicines are necessary for patients who either lack the normal form of a protein or cannot produce it in sufficient quantity.

Such cell growth requires a complex environment containing a mixture of nutrients, including sugars, amino acids, vitamins, minerals, and growth factors such as insulin. Further, except for certain cell types in blood, cells derived from tissues are anchorage-dependent, meaning they do not grow as free-floating individual cells. Therefore, after being released from the tissue environment, cells require a surface on which they can adhere, otherwise they will fail to survive and divide.

After attachment, cells grow and expand onto empty surfaces until the entire surface is covered in a monolayer that is one cell thick. At this point, they stop dividing and reach a state called contact inhibition, at which point the cells need to be detached from the surface and reattached to a surface large enough for growth to resume. This cycle of attachment, cell expansion, and detachment may need to be repeated many times, with each cycle comprised of multiple cell divisions.

Methods of culturing adherent cells inside conventional microfluidics are known, for example, in organ-on-chip applications. In particular, there are existing plate-based workflows that attempt to culture adherent cells. However, in order to achieve cell growth on such platforms, it is necessary to controllably introduce contact between the droplet contents and some kind of culturing region on a chip device, which is complex to achieve in any conventional droplet handling microfluidics platform.

There is some limited literature on culturing adherent cells on digital microfluidics. For example, Barbulovic-Nad et al, in “A microfluidic platform for complete mammalian cell culture”, describe using conventional electrowetting in a lab-on-a-chip platform with an array of electrodes to implement mammalian cell culturing. However such platforms are limited in the number of cells they can manipulate simultaneously due to the large size of the fixed electrodes used for actuation.

Furthermore, with fixed electrode locations and sizes, the flexibility and adaptability of such systems is limited.

The present disclosure provides an apparatus and associated methods for adherent cell culture in which adherent (mammalian) cells are cultured from an emulsion of aqueous microdroplets in oil, and wherein the actuation mechanism for manipulating the cell-containing microdroplets on the surface of a microfluidic chip, and controlling attachment to and detachment from that surface, is optically mediated electrowetting (oEWOD).

The disclosed apparatus thus advantageously allows for the manipulation of microdroplets across a wide range of sizes, and being digitally controlled, provides for dynamically re-programmable operational steps. The microfluidic substrates of the apparatus have no patterned electrodes, removing several complex low-yield fabrication steps and simplifying the electrical interconnections in comparison to conventional approaches. Device failures caused by dielectric breakdown between neighbouring electrodes are also eliminated thereby.

The resulting device structure thus permits more elaborate and integrated workflows compared to conventional approaches, such as independent control of the carrier phase and the droplets, as well as allowing for a greater density of droplets to be controlled across regions of the microfluidic chip surface.

Methods for patterning of the microfluidic chip surface are also provided such that target regions of the chip surface are functionalised to, in conjunction with the disclosed oEWOD actuation mechanism, promote cellular attachment and proliferation to enable controlled growth of target mammalian cells.

### SUMMARY OF INVENTION

According to an aspect of the present invention, a device is provided for manipulating microdroplets, the device comprising a microfluidic chip adapted to receive and manipulate microdroplets dispersed in carrier fluid flowing along pathways on a surface of the chip, wherein the microdroplets are manipulated using an optically-mediated electrowetting (oEWOD) force, and characterised in that the surface of the chip comprises a coating structure configured to allow controlled attachment and/or detachment of adherent cells contained within the microdroplets by application of the oEWOD force.

In some embodiments, the coating structure is formed on the surface of the chip to create one or more wetting areas of the chip configured to facilitate cell adhesion.

In some embodiments, the coating structure may comprise one or more of the following: a polypeptide, collagen, laminin, matrigel, hydrogel or polystyrene.

In a preferred embodiment, the coating structure comprises Polystyrene. In other embodiments, the coating structure comprises at least one of Polylysine, (3-Aminopropyl) trimethoxysilane (APTMS) or Aminopropyltriethoxysilane (APTES)), Collagen, Laminin and Silicon dioxide.

In some embodiments the coating structure comprises one of Bovine Serum Albumin (BSA), Polylysine, Collagen, and Laminin, and forming the coating structure comprises wetting the chip with an aqueous solution comprising said compound such that the compound spontaneously, non-covalently adheres to the underlying surface.

In some embodiments, the surface of the chip comprises a coating structure having one or more hydrophilic patches or regions, in which the coating structure is configured to allow controlled attachment and/or detachment of adherent cells contained within the microdroplets by application of the oEWOD force.

In some embodiments, the coating structure comprises one or more regions or patches that are hydrophobic and/or one or more regions or patches that are hydrophilic. The hydrophilic patches provided on the coating structure can be suitable for cell attachment. The one or more hydrophilic patches can be surrounded by a hydrophobic coating.

An example of a hydrophobic coating is an anti-fouling layer. The anti-fouling coating can be provided in between the hydrophilic patches to give a hydrophobic surface for oEWOD to occur.

In some embodiments, the hydrophilic coating structure may comprise one or more of the following sequences; Gly-Arg-Gly-Asp-Ser (GRGDS), Arg-Gly-Asp (RGD) or Gly-Arg-Gly-Asp-Ser-Pro (GRGDSP). These sequences are short polypeptide sequences which are preferred as it minimise unwanted interferences on the surface of the chip. The surface of the device can be partially coated with the polypeptide to form hydrophilic patches which facilitate cell adhesion. Coating a surface with a short polypeptide to the whole device would provide a hydrophilic surface of the whole device, which would be poor for oEWOD.

To form a coating structure with good compatibility with the oEWOD substrate, an intermediate silane or equivalent is required with an appropriate hydrophilic functional group for the peptide such as RGD to attach to. Without this intermediate linker, the polypeptide sequences would have poor compatibility with the oEWOD device such that the polypeptide would either attach poorly or eventually float away. Bulk coating with the hydrophilic functionalised silane would render the entire surface hydrophilic which is poor for oEWOD.

In some embodiments the coating structure comprises a layer of BSA coupled to the surface via a chemical linker. In embodiments where the underlying surface exposes a layer of aluminium oxide, the chemical linker comprises 16-phosphonohexadecanoic acid or 3-Aminopropylphosphonic acid or any suitable ω-phosphonocarboxylic acids coupled to alkane chain linkers comprised of 3 to 16 (or more) methylene groups. In embodiments where the underlying surface exposes a layer of silicon dioxide, the chemical linker comprises (3-Aminopropyl)trimethoxysilane or a suitable aminoalkylsilane coupled to an alkane chain comprised of 2-6 methylene groups. Other suitable examples include but is not limited to 3-(Triethoxysilyl)propylsuccinic anhydride. In some embodiments, coupling the protein to the aforementioned chemical linkers is done by simultaneously exposing both the BSA and the surface to N-(3-Dimethylaminopropyl)-N′-ethylcarbodiimide hydrochloride (EDC) such that covalent bonds form between the protein groups and the surface. Alternatively, in some embodiments, a covalent bond is formed by first activating the surface using EDC in presence of N-Hydroxysulfosuccinimide sodium salt (sulfo-NHS), and then introducing the BSA in a subsequent step. Alternatively, such covalent bonds can be formed without the use of EDC, for example by using succinimidyl ester or succinic anhydride terminated linkers. In some embodiments the BSA is substituted for another appropriate protein such as collagen, laminin or fibronectin. In other embodiments the BSA is substituted with a mixture of appropriate proteins as detailed above.

In other embodiments, the coating structure comprises Silicon dioxide, and forming the coating structure comprises one of sputtering, atomic layer deposition or thermal evaporation thereof.

In some embodiments, the microfluidic chip of the present invention comprises oEWOD structures comprised of:
a first composite wall comprised of:
   a first substrate
   a first transparent conductor layer on the substrate, the first transparent conductor layer having a thickness in the range 70 to 250 nm;
   a photoactive layer activated by electromagnetic radiation in the wavelength range 400-1000 nm on the conductor layer, the photoactive layer having a thickness in the range 300-1500 nm and
   a first dielectric layer on the photoactive layer, the first dielectric layer having a thickness in the range 30 to 160 nm;
a second composite wall comprised of:
   a second substrate;
   a second conductor layer on the substrate, the second conductor layer having a thickness in the range 70 to 250 nm and
   optionally a second dielectric layer on the second conductor layer, the second dielectric layer having a thickness in the range 30 to 160 nm or 120 to 160 nm
wherein the exposed surfaces of the first and second dielectric layers are disposed less than 180 μm apart to define a microfluidic space adapted to contain microdroplets;
an A/C source to provide a voltage across the first and second composite walls connecting the first and second conductor layers;
at least one source of electromagnetic radiation having an energy higher than the bandgap of the photoactive layer adapted to impinge on the photoactive layer to induce corresponding virtual electrowetting locations on the surface of the first dielectric layer; and
means for manipulating the points of impingement of the electromagnetic radiation on the photoactive layer so as to vary the disposition of the virtual electrowetting locations thereby creating at least one electrowetting pathway along which the microdroplets may be caused to move.

In some embodiments, the first and the second dielectric layers may be composed of a single dielectric material or it may be a composite of two or more dielectric materials. The dielectric layers may be made from, but is not limited to, Al2O3 and SiO2.

In some embodiments, a structure may be provided between the first and second dielectric layers. The structure between the first and second dielectric layers can be made of, but is not limited to, epoxy, polymer, silicon or glass, or mixtures or composites thereof, with straight, angled, curved or micro-structured walls/faces. The structure between the first and second dielectric layers may be connected to the top and bottom composite walls to create a sealed microfluidic device and define the channels and regions within the device. The structure may occupy the gap between the two composite walls.

According to another aspect of the present invention, a surface coating structure for a device is provided, the surface coating structure being configured to allow the adhesion of adherent cells whilst retaining compatibility with an optical electrowetting structure substrate.

In some embodiments, there is provided an intermediate functional molecule to provide compatibility between the oEWOD surface and the cell. The intermediate functional molecule aids the attachment of cells onto the surface of the device. Poor compatibility would result in the loss of hydrophilic region integrity of the coating structure for the device and would subsequently result in the eventual cell dissolution. Without an intermediate functional molecule it would be difficult functionalising the electrode bearing surface to present a hydrophobic surface for drop movement without hindering adhesion of the cell.

In some embodiments, the adherent cells can be in their native adherent state. Unless otherwise specified, the term “native adherent state” as defined herein is referred to the physical and/or chemical properties of an adherent cell in its adherent state where it is capable of proliferation and adopts a stable phenotypic expression state.

In a preferred embodiment, the coating structure comprises polystyrene spin-coated on the chip surface from a solvent solution such as toluene or acetone. In other embodiments, the coating structure comprises patterned plasma oxidised regions of the target surface.

According to another aspect of the present invention, a method of forming a coating structure on a surface of a microfluidic chip comprising an oEWOD active stack is provided, the method comprising: depositing a layer of polystyrene on the surface; depositing a layer of photoresist on the polystyrene; exposing the resist via photomask; developing the photomask to reveal a negative image of one or more target regions, such that target regions remain protected by the photoresist; applying a first solvent to remove exposed areas of polystyrene; applying a second solvent to remove the remaining photoresist covering the target regions.

According to another aspect of the present invention, a method of forming a coating structure on a surface of a microfluidic chip comprising an oEWOD active stack is provided, the method comprising: depositing a layer of photoresist on the surface; exposing the resist via photomask; developing the photomask to reveal one or more target regions; coating or activating the target regions; and lifting off the remaining photomask.

In some embodiments, coating the target regions comprises depositing APTMS on the target regions from liquid phase, using masking to protect regions which have previously been functionalised with fluorosilane from vapour phase.

In some embodiments, the method further comprises, prior to depositing the photoresist on the surface, depositing spin-coated polystyrene on the surface, and coating the surface comprises exposing the target regions to UVO or plasma activation, leaving the unexposed polystyrene un-activated.

In some embodiments, the method further comprises a pre-treatment step of incubating the target regions with a fouling reagent to form a fouling layer and promote culture growth and adhesion of target cells within the device.

In some embodiments, the fouling agent comprises Fetal Bovine Serum. In other embodiments, the fouling agent comprises a standard growth medium such as: F12 growth media, RPMI medium, DMEM, and Opti-MEM. In other embodiments, the fouling agent comprises one of: Green fluorescent protein, Bovine serum albumin, Fibronectin, Collagen, Laminin, Chitin, Matrigel, Hydrogel, and Elastin.

In some embodiments, incubating the target regions with fouling reagent to form the fouling layer is performed subsequent to forming the coating structure.

The application of fouling reagents to the chip surface promotes cell culture by providing a bio-compatible attachment point for the incubated cells which is a close mimic of their natural attachment substrate, such as connective tissue in the body.

In some embodiments, instead of using the fouling agents to spontaneously attach to the surface, the fouling agents are covalently coupled to the surface using a chemical linker.

In some embodiments, the temperature of the cell environment may be controlled to encourage cell detachment form the surface of the target region. For example the chip temperature may be lowered by switching off a heating mechanism and/or cooling the chip surface using a peltier cooler. Such cooling mechanisms may trigger a stress response of cell detachment and may be particularly applicable in assays where detachment proteases/release reagents cannot be used.

The present invention thus provides an integrated platform where automated on-chip operations for screening, sorting, and repeated culturing cycles of adherent cells including attachment, detachment and reattachment, can be performed in the same environment. In contrast, conventional methods require manual handling of cells and repeated transfer of cells to different environments for performing different operations.

Conventional plate-based methods also often have high percentage of “empty” wells during analysis. The on-chip sorting and discarding operations enabled by the oEWOD microfluidic chip of the present invention provide greater efficiency for performing assays.

The small volumes of the microdroplets and the small numbers of cells required per colony to perform assays in an on-chip environment reduces the length of time required for a sufficient number of adherent cells to be cultured.

There is no need to freeze/store cells between assays carried out on the device of the present invention, since cultures can simply be stored on-chip and cells for the next assay can be selected and removed from the colony.

The formation of the coating structure on the surface of the microfluidic chip comprising an oEWOD active stack as disclosed in any aspects of the present invention may be configured to allow controlled attachment and/or detachment of adherent cells contained within the microdroplets by application of the oEWOD force.

### FIGURES

The present invention will now be described, by way of example only, with reference to the accompanying figures in which:

FIG. 1 shows an example configuration of a microfluidic chip comprised of a microdroplet preparation zone and a microdroplet manipulation zone;

FIG. 2A shows a first part of an example workflow for carrying out a method according to the present invention, where cells are adhered to a surface;

FIG. 2B shows a second part of the example workflow for carrying out the method according to the present invention, where cells are detached from the surface; and

FIG. 3 shows an example configuration for carrying out the method of the present invention on a microfluidic chip;

### DETAILED DESCRIPTION

In order to further explain various aspects of the present disclosure, specific embodiments of the present disclosure will now be described in detail in conjunction with the accompanying drawings.

The present invention provides apparatus and associated methods for growing adherent cell cultures by introducing deliberate droplet wetting regions onto a microfluidic chip comprising oEWOD active stack, and using the oEWOD actuated contact angle change to manipulate adherent cell-containing microdroplets to reversibly control the wetting on and off said surface.

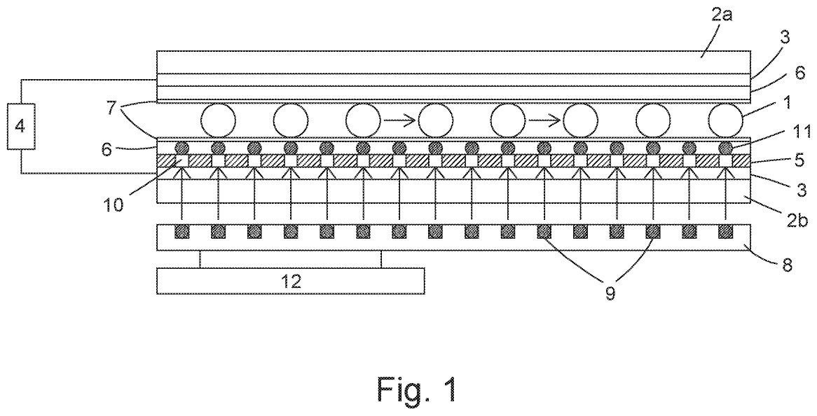

Referring to FIG. 1, an example configuration of a microfluidic chip comprising an oEWOD stack suitable for carrying out methods according to the present invention is illustrated.

The example device is suitable for the manipulation of aqueous microdroplets 1 having been emulsified into a fluorocarbon oil, having a viscosity of 1 centistokes or less at 25° C. and which in their unconfined state have a diameter of less than 100 μm (e.g. in the range 20 to 80 μm).

The oEWOD stack of the device comprises top 2a and bottom 2b glass plates each 500 μm thick coated with transparent layers of conductive Indium Tin Oxide (ITO) 3 having a thickness of 130 nm. Each of the layers of conductive Indium Tin Oxide (ITO) 3 is connected to an A/C source 4 with the ITO layer on bottom glass plate 2b being the ground. Bottom glass plate 2b is coated with a layer of amorphous silicon 5 which is 800 nm thick. Top glass plate 2a and the layer of amorphous silicon 5 are each coated with a 160 nm thick layer of high purity alumina or Hafnia 6 which are in turn coated with a monolayer of poly(3-(trimethoxysilyl)propyl methacrylate) 7 to render the surfaces of the layer of high purity alumina or Hafnia 6 hydrophobic.

Top glass plate 2a and the layer of amorphous silicon 5 are spaced 8 μm apart using spacers (not shown) so that the microdroplets undergo a degree of compression when introduced into the device cavity. An image of a reflective pixelated screen, illuminated by an LED light source 8 is disposed generally beneath bottom glass plate 2b and visible light (wavelength 660 or 830 nm) at a level of 0.01 Wcm2 is emitted from each diode 9 and caused to impinge on the layer of amorphous silicon 5 by propagation in the direction of the multiple upward arrows through bottom glass plate 2b and the layer of conductive Indium Tin Oxide (ITO) 3.

At the various points of impingement, photoexcited regions of charge 10 are created in the layer of amorphous silicon 5 which induce modified liquid-solid contact angles on the layer of high purity alumina or Hafnia 6 at corresponding electrowetting locations 11. These modified properties provide the capillary force necessary to propel the microdroplets 1 from one electrowetting location 11 to another. LED light source 8 is controlled by a microprocessor 12 which determines which of the diodes 9 in the array are illuminated at any given time by pre-programmed algorithms.

Further specific details of microfluidic chips suitable for carrying out the methods of the present invention may be found in our published patent WO 2018/234445, which is herein incorporated by reference.

The device of the present invention also provides for implementing environment controls suitable for the adherent cell conditions such as: controlled temperature, regions of different flow, controlling the carrier fluid to continuously feed cultured cells a supply of nutrients, and control of the local gas concentration in the carrier fluid surrounding the cultured cells.

For example, the adherent cell culture may be located in a region of low flow and surrounded by regions of faster flow that contain and supply nutrients and chemicals to the culture to encouraging growth.

Also provided herein are surface coating structures for encouraging adhesion of cells contained in microdroplets. The coating structures being configured to cause target regions of the chip to be suited to transport and adherence without adversely affecting the precision of the microdroplet manipulation of the oEWOD chip.

Methods of patterning surface coating structures according to the present invention are also provided herein.

In some embodiments the coating structure may be formed across the entire surface of the microfluidic chip. In other embodiments only a part of the surface of the microfluidic chip may be patterned with the coating structure.

Example coating structures and coating structure formation methods that have been screened experimentally and determined to be viable for cell adhesion and proliferation (specifically, using Chinese Hamster Ovary (CHO) cells) and for oEWOD chip manipulation include: the deposition of APTMS, the deposition and selective activation of spin-coated Polystyrene, and the selective removal and deposition of Polystyrene by application of orthogonal solvents. Each of these methods is described below in greater detail.

Surface coatings that have been screened and found to work for both cell adhesion and oEWOD manipulation include Silicon substrate, Indium Tin Oxide (ITO), amorphous silicon, Alumina, Silicon dioxide, APTMS, and Polystyrene spin-coated from a solvent solution each of which may be deposited via any of sputtering, evaporation, and atomic layer deposition.

During the formation of one example coating structure, APTMS culturing patches were formed by depositing a layer of photoresist onto the surface of a standard oEWOD active stack of a microfluidic chip (the oEWOD stack being configured as described above). A photomask was then used to expose the resist to light, the photomask was developed and lifted-off to leave only the target regions of the surface exposed. At this point the APTMS coating was deposited onto the target regions in from liquid phase and the remaining resist was removed, resulting in an APTMS coating structure being formed only on the target regions.

In the formation of another example coating structure, Polystyrene coating patches with selective activation were formed by depositing spin-coated polystyrene onto the surface of the microfluidic chip. Subsequently, a layer of photoresist was deposited on the polystyrene and a photomask was applied to expose target regions, followed by development and lift-off of the photomask to leave only the target regions of Polystyrene exposed Subsequently, ultraviolet optical activation was applied to the exposed regions, and the remaining photomask was removed to leave patches of activated polystyrene in the target regions, surrounded by un-activated Polystyrene in the other regions.

In yet another example, a coating structure was formed by selective Polystyrene deposition with orthogonal solvents. In this example, a layer of Polystyrene was deposited on the microfluidic chip surface, followed by a layer of photoresist on the Polystyrene surface. The resist was exposed via photomask, the photomask being developed and lifted off to leave target regions exposed. Tested solvents include aqueous solution of sodium hydroxide and potassium hydroxide, however it is anticipated that a wide range of basic solutions will be applicable.

Once a surface coating structure has been formed according to one of the above methods, the target regions which have been coated to encourage adherent cell culturing may also be pre-treated prior to beginning the process of cell culturing.

For example, deliberate fouling of the target regions performed by incubating the target regions in Fetal Bovine serum was shown to have a positive effect on the attachment and proliferation of target cells to target regions of the oEWOD chip surface. This fouling process may be carried out using emulsified microdroplets, whereby droplets containing the fouling material are driven to the target region and deliberately allowed to foul the target region.

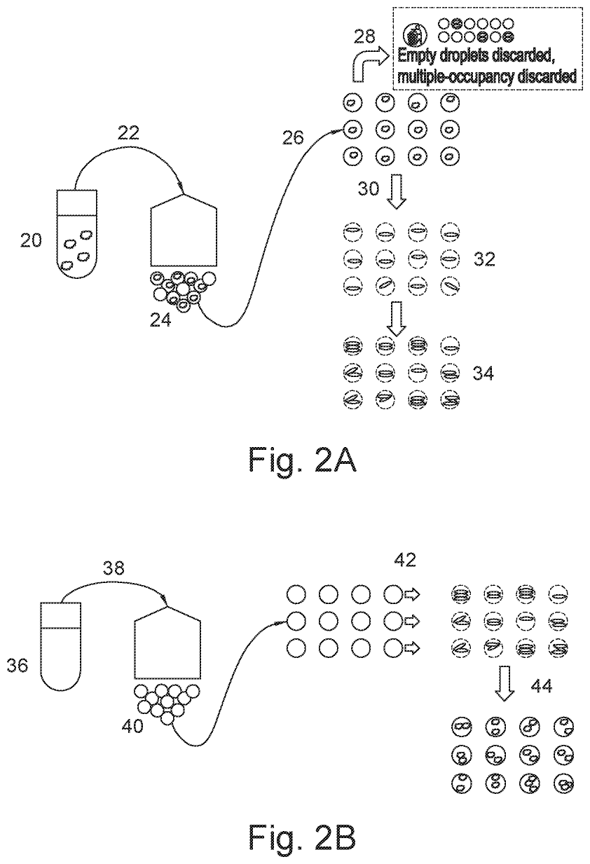

Referring to FIGS. 2A and 2B, an example workflow for carrying out an assay using the method of the present invention will now be described.

FIG. 2A illustrates a first part of the experimental workflow, wherein a sample of transfected adherent cells is emulsified into a plurality of cell-containing microdroplets and which are caused to adhere to a target region of a microfluidic chip.

In a first step 20, the sample of transfected cells are suspended in an aqueous solution. Typically the solution comprises an oil such as, for example, HFE-7500, HFE-7700, FC-40, FC-70. Such oils are chosen to contain a suitable fluorinated surfactant such as RAN-008, Picosurf 1, Picosurf 2, or dSurf. In a second step 22 the solution is then emulsified into a plurality of first microdroplets. Some of the first microdroplets contain cells and some do not.

In a third step 24 the microdroplets are loaded onto a microfluidic chip, such as the microfluidic chip comprising an oEWOD stack structure as described above. The microfluidic chip is then configured to sort the first microdroplets 26 into cell containing and empty microdroplets, with the empty microdroplets being discarded 28. The sorting may be performed by optical inspection of each Microdroplet and the droplets may be manipulated along the surface of the microfluidic chip via oEWOD induced forces.

By the same oEWOD mechanism, the remaining first microdroplets are manipulated into position 30 on the surface of the chip. For example, the remaining first microdroplets may be caused to move to one or more target regions of the chip surface which have been prepared with a coating structure to encourage adhesion of adherent cell cultures as described above. Once in position at the target region, electrowetting manipulation causes the remaining first microdroplets to expose the contained cells to the surface of the one or more target regions such that cells adhere to the surface 32, the cells are then allowed to proliferate in a culturing step 34. In some embodiments, the culturing step requires the cells to be held in position at the one or more target regions for 5 to 15 hours. In other embodiments, the cells are held in position for longer, such as up to 72 hours.

Referring to FIG. 2B, a second part of the experimental workflow is illustrated, wherein a release reagent for encouraging cell detachment from the target region of the surface is emulsified into a set of second microdroplets and used to cause the cultured cells to detach from the microfluidic chip.

In a first step 36 a release reagent is suspended in an aqueous solution. The release reagent may comprise one of Accutase, Trypsin, or a chelating agent such as Ethylenediaminetetraacetic acid (EDTA). Similarly to the first workflow, the suspended solution typically comprises an oil such as, for example, HFE-7500, HFE-7700, FC-40, FC-70. Such oils are chosen to contain a suitable fluorosurfactant such as RAN-008, Picosurf 1, Picosurf 2, or dSurf. In a second step 38 the solution is emulsified into a plurality of second microdroplets.

In a third step 40 the second microdroplets are loaded onto the same microfluidic chip as the first microdroplets, and are positioned adjacent to the first microdroplets containing the adhered cells on the microfluidic chip surface. The second microdroplets may, for example, be caused to pair up with the first microdroplets in two paired microdroplet arrays. The second microdroplets are then caused to merge 42 with the first microdroplets to form merged microdroplets and introducing the Trypsinizing reagent to the cell colonies contained in the first microdroplets.

Once merged, the combination of the Trypsinizing reagent interaction and the application of oEWOD dewetting forces 44 pulling each merged microdroplet to away from the target region surface causes the adhered cells to detach from said surface, allowing the cells to be returned to suspension.

The above described workflow can allow the culturing cycle to be repeated by, subsequent to detachment, re-adhering the cells to a wider area of the target region once they have reached their proliferation limit.

Such cycles can be repeated as many times as necessary until a sufficient number of clones have been obtained to perform desired assays, which can be done on-chip or off-chip.

Some example assays have been performed on such cultured cells on-chip, such as, for example, the introduction of a fluorescent reporter dye to the cultured cells. Assays comprising introduction of an additional reagent may be performed in a similar manner to the introduction of release reagent as described above, wherein the reagents are introduced in the form of emulsified aqueous microdroplets and merged with the cell-containing droplets which are already on chip.

Other example assays that could be performed on the cultured cells on-chip include: the introduction of a reporter bead, the introduction of a FRET reporter, the imaging of an endogenously expressed reporter, microscopic cell morphology measurements, lysis of the cultured cells, genetic detection assays such as PCR, isothermal amplification or fluorescence in-situ hybridisation, and DNA sequencing preparation. Alternatively the detached cells can simply be flowed off-chip for further analysis.

Other example experiments that have been performed include the growth of CHO cells in microdroplets on microfluidic devices that have been provided with a uniform coating of UVO-activated Polystyrene on a surface of an oEWOD stack, wherein the microdroplets have were caused to wet target regions of the device surface using oEWOD forces and CHO cells were adhered to the target regions then detached by adding Accutase reagent.

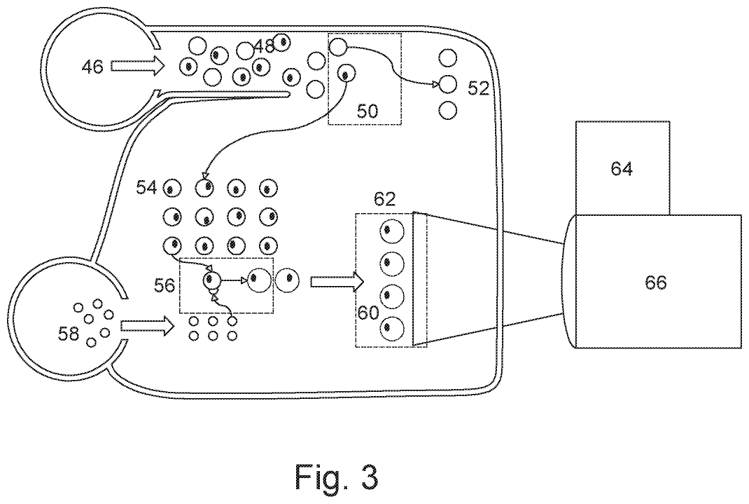

FIG. 3 illustrates an example workflow according to an aspect of the present invention being carried out on the surface of an oEWOD microfluidic chip device.

The view illustrated is of the surface of the oEWOD microfluidic chip, which is configured to manipulate various microdroplets, containing respective emulsified cell samples and reagents, between different locations on the surface.

It is upon such a surface that the coating structure and fouling layer according to the present invention may be formed to provide target regions of the surface with the additional functionality of enabling controlled adherence and detachment of mammalian cells contained within manipulated microdroplets.

At the beginning of the workflow, fluid inlet 46 admits an emulsion 48 of a mixture of empty and cell-containing first microdroplets in a fluorocarbon oil carrier fluid.

These first microdroplets are then transferred by means of OEWOD structures of the chip to a sorting zone 50 where they are sorted into those which are empty 52 and those which contain cells 54. Thereafter each of the cell-containing microdroplets 54 are transferred to merging zone 56 which in this example is a target region of the chip surface which has already been provided with a coating structure for promoting mammalian cell adherence and, optionally, a fouling layer providing a bio-compatible attachment point for the contained cells which is a close mimic of their natural attachment substrate.

The cells are held in place on the target region for a predefined period of time under conditions which promote cell growth and division within each, forming a colony of adhered cells on the surface within the first microdroplets.

At the end of this period, a second inlet 58 admits second microdroplets. The second microdroplets may be an emulsion of a fluorocarbon oil and a release reagent for encouraging detachment of the cells contained in the first microdroplets as described above in relation to FIG. 2B. The release reagent may be chosen from Accutase, Trypsin, Citrate buffer or a chelating agent such as Ethylenediaminetetraacetic acid (EDTA).

The second microdroplets are then merged with the cell-containing first microdroplets 52 at merging zone 56 to form merged microdroplets 60 and left for a predefined time. For example, once merged the merged microdroplets may be left to incubate for between 5 and 30 minutes at a temperature of 37° C. During incubation the droplets are monitored via an optical detection system checking for signs that the attached cells are releasing, such as the cell profiles becoming globular.

At this point in the workflow the cells contained in the merged microdroplets, each droplet now containing a plurality of cells, may be manipulated according to the needs of particular sampling assays in any number of ways. Such manipulation may comprise altering the electrowetting conditions for the microdroplets such that the microdroplets de-wet or partially de-wet from the surface. The term “de-wet” as used herein refers to the change in contact angle between the droplet and the chip surface such that the droplet is pulled away from the surface.

The oEWOD forces may also be used to agitate and “stir” the droplets to disperse the cells contained within; this has the effect of separating cells which may have become attached to each other and ensuring an even spatial distribution for imaging the cells. The forces may be used to stretch and elongate the droplets to break off smaller, daughter droplets if it is desired to assay a single cell from a cultured colony. This process may be aided somewhat by the mother droplet remaining wetted or partially wetted to the surface of the target region. The daughter droplets may then be inspected for cell occupancy and, if the desired cell distribution is not achieved, the droplets may be re-merged and split once more.

Optionally, a plurality of third microdroplets containing a fluorescence reporter system selective for a cell type of interest may also be introduced and merged with the first and second microdroplets at merging zone 56. The merged microdroplets 60 can then be transferred by means of OEWOD structures to optical window 62 where a fluorescence signal characteristic of the reporter system is detected using an optical detection instrument 64 comprised of an LED light source, a photodetector and a microprocessor. Optical detection instrument 64 is partially combined with an optical manipulation projector 66.

Optionally, the release reaction between the target cells and the release reagent may be allowed to self-quench through depletion of the release reagent or, for example, by the addition of a protein substrate such as serum. Other quench mechanisms might be suitable too.

Subsequent to the above-described manipulation, some subset of the cells may be returned to the target regions and allowed to re-adhere for further culturing. The subset chosen for retention may depend on the result of an assay run on the sampled droplets.

As used herein, the term “fouling layer” may refer to a substance such as a biomolecule which may be absorbed onto a surface.

As used herein, the term “coating structure” may refer to a substance, such as Polystyrene, APTMS, or Silicon dioxide, which is covalently bonded to a surface.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments. It is not limited to the disclosed embodiments and that alternative embodiments could be constructed without departing from the scope of the invention as defined in the appended claims.

1-24. (canceled)

25. A device for manipulating microdroplets, the device comprising a microfluidic chip adapted to receive and manipulate microdroplets dispersed in carrier fluid flowing along pathways on a surface of the chip, wherein the microdroplets are manipulated using an optically-mediated electrowetting (oEWOD) force wherein the surface of the chip comprises a coating structure configured to allow controlled attachment and/or detachment of adherent cells contained within the microdroplets by application of the oEWOD force.

26. The device of claim 25, wherein the coating structure is formed on the surface of the chip to create one or more wetting areas of the chip configured to facilitate cell adhesion.

27. The device of claim 25, wherein the coating structure comprises silicon dioxide.

28. The device of claim 25, wherein the coating structure comprises polystyrene.

29. The device of claim 25, wherein the chip of the device consists essentially of:
a first composite wall comprised of:
a first substrate
a first transparent conductor layer on the substrate, the first transparent conductor layer having a thickness in the range 70 to 250 nm;
a photoactive layer activated by electromagnetic radiation in the wavelength range 400-1000 nm on the conductor layer, the photoactive layer having a thickness in the range 300-1500 nm and
a first dielectric layer on the photoactive layer, the first dielectric layer having a thickness in the range 30 to 160 nm;
a second composite wall comprised of:
a second substrate;
a second conductor layer on the substrate, the second conductor layer having a thickness in the range 70 to 250 nm and
optionally a second dielectric layer on the second conductor layer, the second dielectric layer having a thickness in the range 30 to 160 nm or 120 to 160 nm
wherein the exposed surfaces of the first and second dielectric layers are disposed less than 180 μm apart to define a microfluidic space adapted to contain microdroplets;
an A/C source to provide a voltage across the first and second composite walls connecting the first and second conductor layers;
at least one source of electromagnetic radiation having an energy higher than the bandgap of the photoactive layer adapted to impinge on the photoactive layer to induce corresponding virtual electrowetting locations on the surface of the first dielectric layer; and
means for manipulating the points of impingement of the electromagnetic radiation on the photoactive layer so as to vary the disposition of the virtual electrowetting locations thereby creating at least one electrowetting pathway along which the microdroplets may be caused to move.

30. A surface coating structure for a device according to claim 25, the surface coating structure being configured to allow the adhesion of adherent cells whilst retaining compatibility with an optical electrowetting structure substrate.

31. The coating structure of claim 30, wherein the coating structure comprises silicon dioxide deposited on the chip surface through sputtering or evaporation, APTMS deposited in vapour phase.

32. The coating structure of claim 30, wherein the coating structure comprises polystyrene spin-coated on the chip surface from a solvent solution.

33. A method of manipulating adherent cells contained in microdroplets on a surface of a microfluidic chip comprising an oEWOD active stack, the method comprising:
positioning first microdroplets on one or more target regions of the surface, the first microdroplets containing adherent cells;
allowing the cells from the first microdroplets to adhere to the target regions;
introducing second microdroplets to the target regions, the second microdroplets containing a release reagent; and
merging the first microdroplets with the second microdroplets, such that the release reagent causes the cells from the first microdroplets to detach from the target regions.

34. The method of claim 33, wherein the method further comprises applying an optically-mediated electrowetting (oEWOD) force to the merged microdroplets to promote cell detachment.

35. The method of claim 33, wherein the step of allowing the cells from the first microdroplets to adhere further comprises allowing the cells to replicate.

36. The method of claim 35, wherein microdroplets containing replicated cells are split to divide the contained cells between a plurality of daughter droplets.

37. The method of claim 33, wherein the method further comprises washing the target regions to dilute and remove release reagents.

38. The method of claim 33, wherein the method further comprises adding a deactivating reagent to remove release reagents.

39. The method of claim 33, wherein the method further comprises returning the cells to the target regions and allowing them to re-adhere and replicate.

40. The method of claim 33, wherein the release agent comprises at least one of trypsin, citrate buffer, a chelating agent, and Accutase.

41. The method of claim 33, wherein the method further comprises controlling the temperature of the chip to encourage cell detachment.
